(19) United States
(12) Patent Application Publication
(10) Pub. No.: US 2023/0270815 A1
(43) Pub. Date: Aug. 31, 2023

(54) Apoptosis inducing peptide (SSTP1)

(71) Applicant: Rajiv Gandhi Centre For Biotechnology, Kerala (IN)

(72) Inventors: Tessy Thomas MALIEKAL, Kerala (IN); Shyla G, Kerala (IN)

(73) Assignee: Rajiv Gandhi Centre For Biotechnology, Kerala (IN)

(21) Appl. No.: 17/919,130

(22) PCT Filed: Apr. 14, 2021
(86) PCT No.: PCT/IN2021/050371

§ 371 Date: Oct. 14, 2022

(30) Foreign Application Priority Data
Apr. 15, 2020 (IN) 202041016382

(51) Int. Cl. A61K 38/10; A61P 35/00

(52) U.S. Cl. CPC A61K 38/10; A61P 35/00

(57) ABSTRACT

The present invention relates to a peptide SSTP1 that modulates IL6 pathway to induce apoptosis in cells where there is overexpression of IL6Rα (Interleukin 6 Receptor). This is achieved by inhibiting IL6/JAK/STAT pathway and/or by activating JNK/AP1 signal transduction pathway. The sensitivity of the cancer cells to SSTP1 was proportional to IL6Rα levels. Further, SSTP1 kills triple negative breast cancer cells, MDA-MB-231, at a concentration that cause neither hemolysis nor cell death to blood cells. The peptide of the present invention utilizes the elements of IL6 pathway and induces apoptosis in cancer cells. SSTP1 functions by activation of JNK/AP1 pathway with concomitant inhibition of STATs, offering a new therapeutic strategy to treat cancer cells that overexpress IL6Rα.

### SEQUENCE LISTING

The instant application contains a Sequence Listing which has been submitted electronically in ASCII format and is hereby incorporated by reference in its entirety. Said ASCII copy, created on May 8, 2023, is named PUTHRAN-0010_SL.txt and is 902 bytes in size.

### FIELD OF INVENTION

The present invention relates to the field of anti-carcinogenic peptides. It further relates to the field of anti-carcinogenic peptides, which modulates IL6 pathway, specific to induction of apoptosis in cells with significant overexpression of IL6Rα (Interleukin 6 Receptorα). The invention further relates to a composition comprising the peptide and the method of preparation thereof. Further the invention relates to inhibition of IL6/JAK/STAT pathway and/or by activating JNK/AP1 signal transduction pathway by the novel Peptide and its composition thereof. This active induction of apoptosis is considered to be a novel strategy to induce apoptosis in cancer cells.

### BACKGROUND OF THE INVENTION

Cancer is one of the leading causes of death in developed countries. Chemotherapy is one of the major approaches to treat cancer by delivering a cytotoxic agent to the cancer cells. The main problem with the conventional chemotherapy is it being non targeted. The treatment options against cancer also include the use of proteins, Mabs and peptides. The anticancer peptides possess many advantages, such as small size, ease of synthesis and modification, tumor-penetrating ability, and good biocompatibility.

There are several pathways involved in the cancer biology, however some of the prior art discusses the predominant role of IL6 in cancer is its promotion of tumour growth. IL6 binds IL6 receptor (IL6Rα) and the membrane-bound glycoprotein gp130. The IL6/IL6Rα/gp130 complex starts the Janus kinases (JAKs) and signal transducer and activator of transcription 3 (STAT3) or JAK/STAT3 pathway. Overexpression of IL6 and hyper-activation of IL6/IL6R pathway is associated with inflammatory diseases and cancers.

Several studies have shown to generate antibodies that neutralize IL6 or IL6R, and inhibitors that block JAK/STAT pathway are used clinically to treat inflammatory diseases and cancer. However, IL6 inhibitors and inhibition of JAK/STAT activity were not effective to treat triple negative breast cancer.

The present study identified and studied a specific peptide SSTP1 isolated from frog skin secretion which triggers/induces apoptosis in cancer cells. It is also observed that the sensitivity of the peptide is directly proportional to IL6Rα levels.

Our experimental data suggest that the peptide alters the signaling mediated by IL6/IL6Rα/gp130 complex. When treated with the peptide SSTP1, the peptide down-regulates pSTAT3 Y705 phosphorylation, if there is a sustained high pSTAT3 levels. Another important observation identified is the up-regulation of JNK/AP1 apoptotic pathway. The peptide utilizes the components of IL6 pathway and induces apoptosis in cancer cells.

This active induction of apoptosis with the peptide SSTP1 is considered to beanovel strategy for treating cancer cells that shows up-regulation of IL6Rα. Thus this peptide is observed to induce apoptosis in cancer cells overexpressing IL6Rα.

### Objectives of the Present Invention

The primary objective of the present invention is to identify peptide (SSTP1-FLPLLISALTSLFPKLGK (SEQ ID NO: 1)) that can induce apoptosis in cells that overexpress IL6Rα.

It is another objective of the present invention to induce apoptosis in cancer cells, specifically triple negative cancers with the help of SSTP1 peptide.

Another objective of the present invention is to formulate a composition comprising the peptide (SSTP1-FLPLLISALTSLFPKLGK (SEQ ID NO: 1)) in medical/pharmacological conditions where IL6Rα is overexpressed.

Yet another objective of the present invention is to identify and use the peptide SSTP1 in treating disorders where IL6α-overexpression is observed similar to hematological disorders like multiple myeloma, rheumatoid arthritis and various malignancies.

Yet another objective of the present invention is to provide treatment options involving peptide as one of the active ingredient along with the pharmaceutically acceptable peptide stabilizer, carrier and excipients.

Another objective of the invention is to design derivatives of the peptide SSTP1 which could be used as drugs and pharmaceutical compositions.

### SUMMARY OF THE INVENTION

Temporin is a frog-derived peptide. The present invention identified a peptide of temporin family from skin secretions of Indosylvirana aurantiaca, which potentially induced apoptosis in cancer cells. The peptide SSTP1-FLPLLISALTSLFPKLGK (SEQ ID NO: 1) utilizes IL6 pathway and helps in effectively destroying triple negative breast cancer cells, MDA-MB-231 (which has high IL6Rα compared to HSC-4). The IC50 value for MDA-MB-231 and HSC-4 are 4.5 μM and 10.22 μM, respectively. The peptide utilizes the components of IL6 pathway and induces apoptosis in cancer cells. This active induction of apoptosis is considered to be the novel strategy to induce apoptosis in cancer cells where overexpression of IL6Rα is observed. Further, the induction of hemolysis at 5 μM concentration of SSTP1 was only 0.25%, suggesting that this peptide may be safe for in vivo administration. SSTP1 at 4.5 μM did not affect the viability of leukocytes, which express IL6Rα and are the primary mediators of IL6 immunoregulation. The invention further relates to a composition comprising the peptide and the method of preparation thereof.

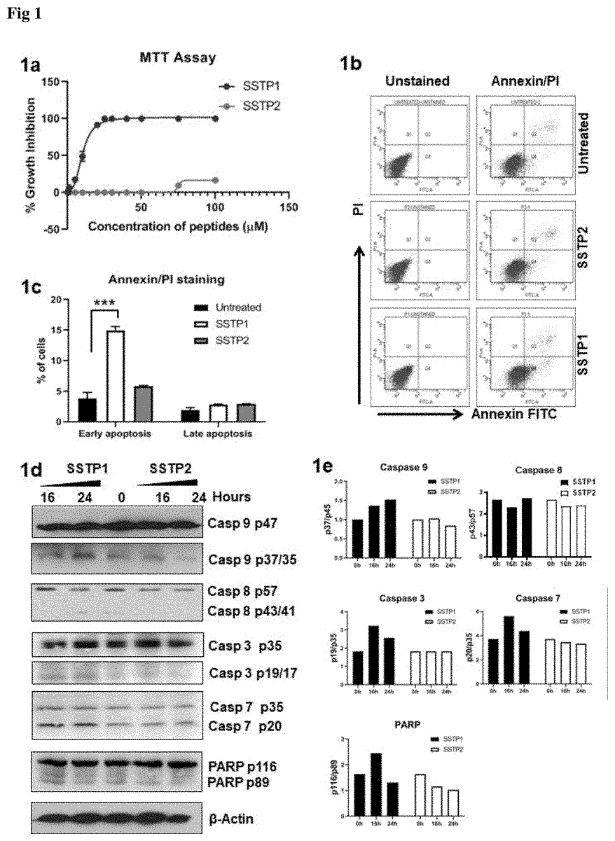

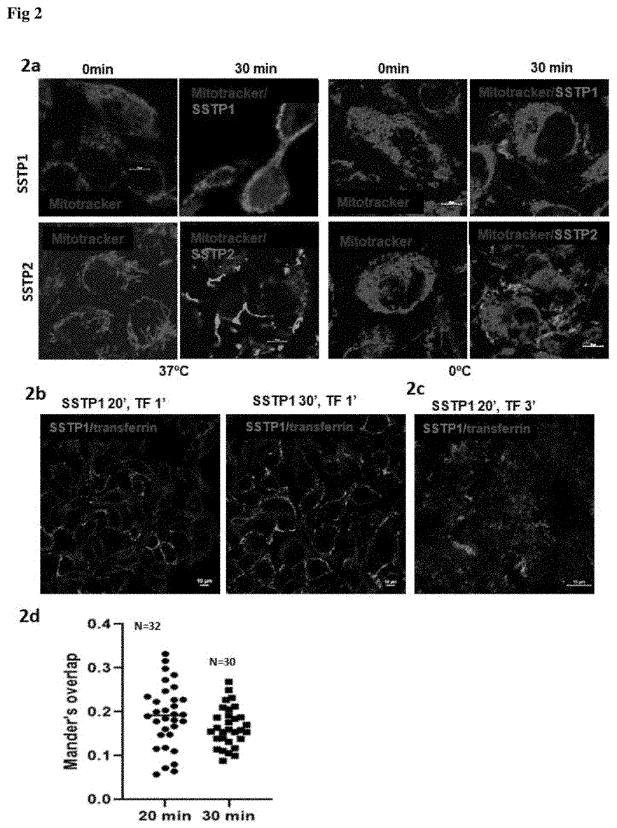

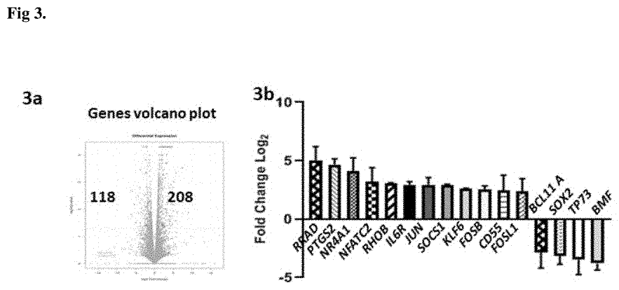

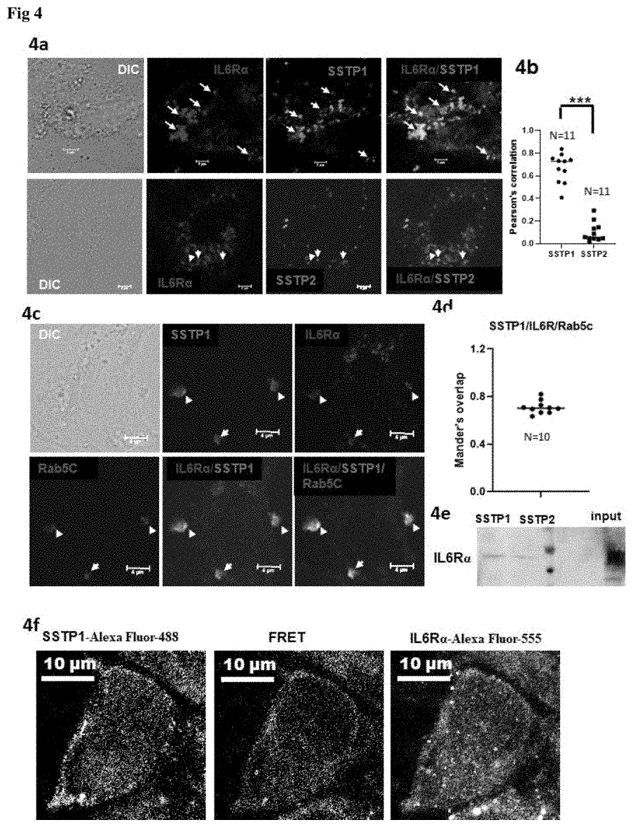

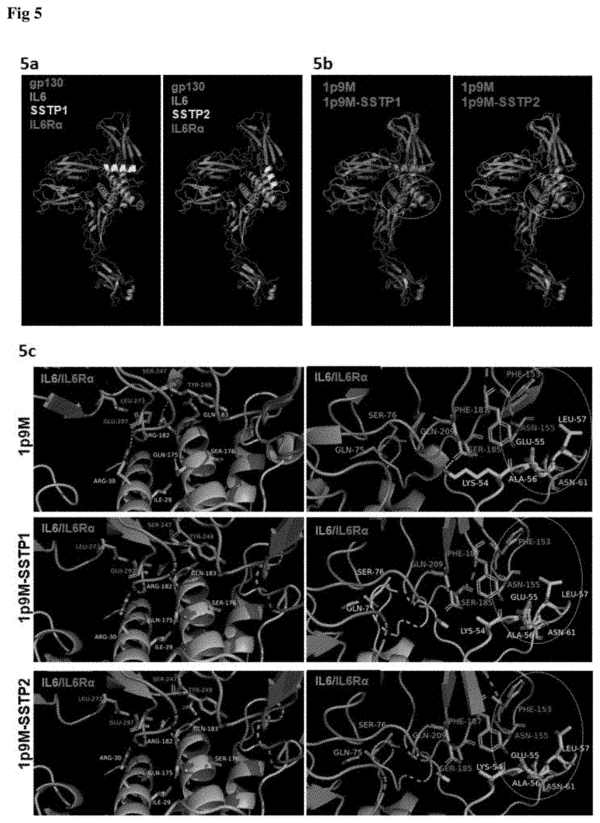

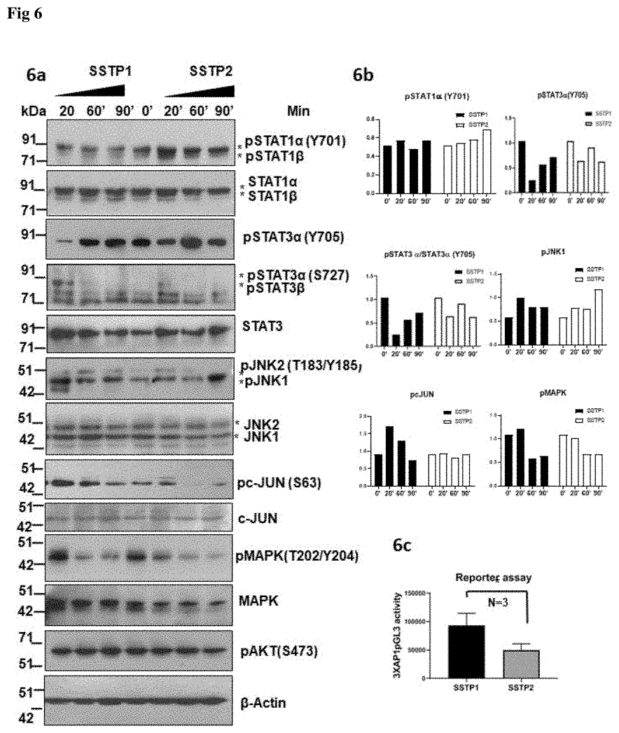

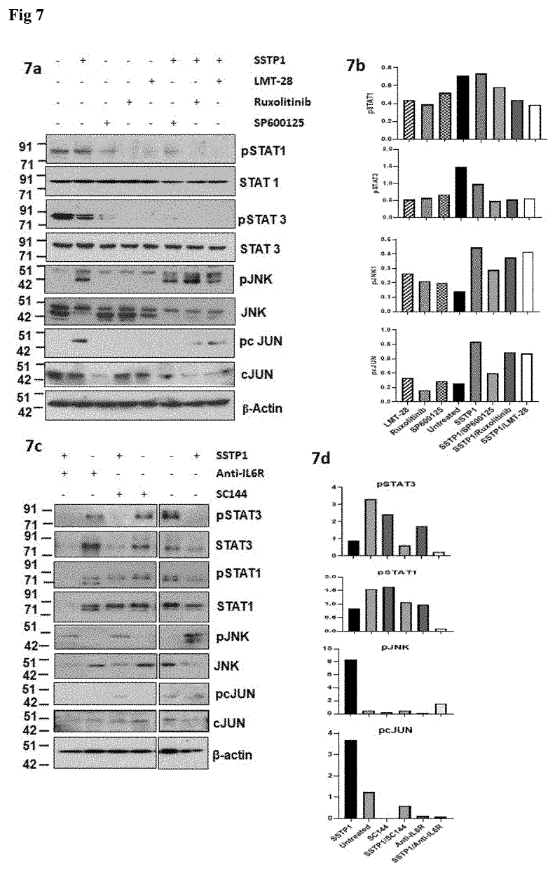

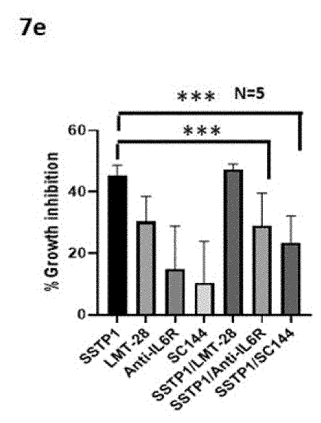

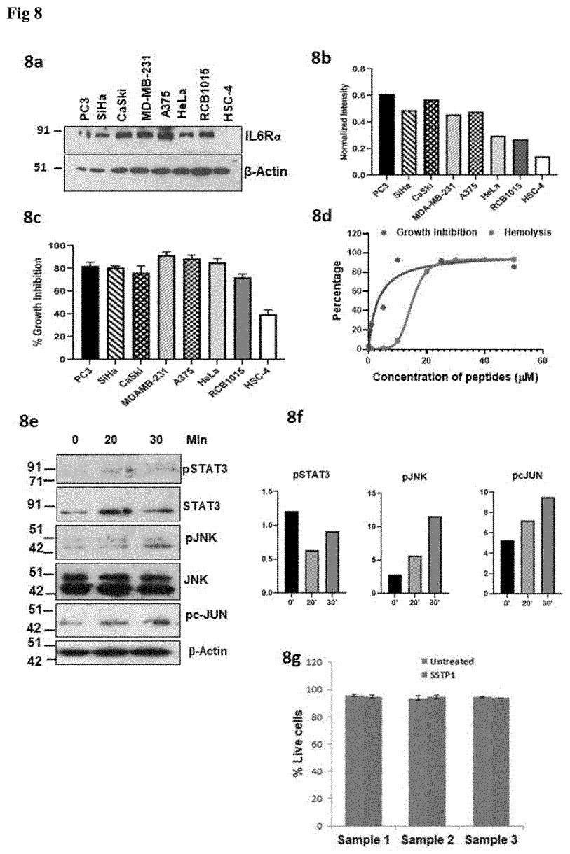

### BRIEF DESCRIPTION OF THE ACCOMPANYING DRAWINGS

| | |
|---|---|
| FIG. 1a | Represents the % inhibition of viability as assessed by MTT |
| | assay. |
| FIG. 1b | Graph representing Annexin/PI Staining of untreated, control |
| | (SSTP2) and SSTP1Vs percentage of cells at early and late |
| | apoptosis.HSC-4 cells were treated with 10 μM SSTP1 and |
| | SSTP2 for 22 h and FACS analysis was performed after |
| | annexin/PI staining. |
| FIG. 1c | Graphical representation of the results of FACS analysis after |
| | Annexin/PI Staining. |
| FIG. 1d | HSC-4 cells were treated with 10 μM SSTP1 and SSTP2 for |
| | indicated time and Western blot was performed to show the |
| | activity or cleavage of caspases. |
| FIG. 1e | Densitometric analysis of the bands. |
| FIG. 2a | Illustrates SSTP1 is internalized by active uptake |
| | HSC-4 cells were treated with 10 μM of Alexa 488 tagged |
| | SSTP1 or SSTP2 for 30 minutes and stained for IL6Rα |
| FIG. 2b, c, d | Illustrates SSTP1 internalization is clathrin-independentHSC-4 |
| | cells were treated with 10 μMof Alexa 488 tagged SSTP1 or |
| | SSTP2 for 30 minutesfollowed by 1 minute treatment of alexa |
| | 568-tagged transferrin. |
| FIG. 3a | Represents the differential expression of genes identified by |
| | RNA-Seq analysis. HSC-4 cells were treated with 10 μM SSTP1 |
| | and SSTP2 for 3 h, and total RNA was isolated to perform |
| | RNA-Seq analysis. |
| FIG. 3b | Confirmation of RNA-Seq results by qPCR |
| FIG. 4a, b | Represents the image showing the binding of SSTP1 to IL6Rαon |
| | cell surface. Pearson's correlation is plotted to show the |
| | colocalization |
| FIG. 4c, d | Endosomal localization of IL6Rαbound SSTP1 as revealed by the |
| | co-localization with endosomal marker, Rab5c. Mander's overlap |
| | is plotted to show the co-expression. |
| FIG. 4e | Pull-down assay to show the binding of biotin-tagged SSTP1 and |
| | SSTP2 to IL6Rα |
| FIG. 4f | FRET analysis to illustrate the binding of SSTP1 to IL6Rαon cell |
| | surface |
| FIG. 5a | Represents the models SSTP1/SSTP2 bound IL6/IL6Rα/gp130 |
| | complex.Molecular docking and dynamic simulation was |
| | performed to identify the binding of SSTP1 and SSTP2 to IL6Rα. |
| FIG. 5b | The peptide bound and unbound IL6/IL6Rα/gp130 complex were |
| | superimposed to visualize the conformational change. The dotted |
| | circles represent the region where conformational change occurs. |
| FIG. 5c | Zoomed images of the interface between IL6 and IL6Rαrevealing |
| | the rearrangement in the active site residues. |
| FIG. 6a | Western blot of HSC-4 cells treated with the peptides |
| | SSTP1 or SSTP2 (10 μM)to show the activation of JNK/AP1 |
| | pathway and inhibition of STAT pathway |
| FIG. 6b | Densitometric analysis was performed and relative |
| | phosphorylation of each molecule was calculated |
| FIG. 6c | A luciferase reporter assay to show activation of AP1 |
| FIG. 7a | HSC-4 cells were treated with SSTP1(10 μM), LMT-28(60 μM), |
| | S-Ruxolitinib(20 μM)orSP600125 (20 μM)as indicated, and |
| | western blot was performed for the indicated molecules |
| FIG. 7b | Densitometric analysis was done for the bands and relative |
| | phosphorylation is plotted. |
| FIG. 7c | HSC-4 cells were treated with SSTP1(10 μM), SC144(40 μM) |
| | and Ant-IL6Rα(10 μg/ml) as indicated, and western blot was |
| | performed after 30 minutes for the indicated molecules. |
| FIG. 7d | Densitometric analysis was done for the bands and relative |
| | phosphorylation was plotted. |
| FIG. 7e | Illustrates the growth inhibition analyzed by MTT assay after 4 h |
| | treatment with SSTP1 (10 μM) with or without LMT-28 (60 μM), |
| | Anti- IL6Rα (10 μg/ml) or SC144 (40 μM) |
| FIG. 8a | IL6Rα levels in different cell lines were analysed by western |
| | blot in comparison to HSC-4 cells |
| FIG. 8b | Illustrates densitometric analysis of the IL6Rα levels |
| FIG. 8c | MTT assay performed with SSTP1 (10 μM) |
| FIG. 8d | Growth inhibition induced in MDA-MB-231 cells and hemolysis |
| | induced in human RBCs when treated with different |
| | concentrations of SSTP1 |
| FIG. 8e | Western blot analysis of indicated molecules when MDA-MB-231 |
| | cells were treated with SSTP1 (4.5 μM) |
| FIG. 8f | Illustrates the denstiometric analysis was done for the bands (c) |
| | and relative phosphorylation is plotted. |
| FIG. 8g | Illustrates SSTP1 do not induce cell death in human leukocytes |
| | when treated with SSTP1 (4.5 μM). Trypan blue exclusion was |
| | performed to assess cell death. |

### DETAILED DESCRIPTION

The present invention relates to a peptide obtained from frog skin secretion, which is capable of inducing apoptosis in cells where there is overexpression of IL6Rα (Interleukin 6 Receptor), which is a Protein Coding gene. In specific the present invention relates to the peptide SSTP1-FLPLLISALTSLFPKLGK (SEQ ID NO: 1). The invention further relates to a composition comprising the peptide and the method of preparation thereof.

The present invention provides evidences and data to support that SSTP1 induces cell death by mitochondrial pathway in oral cancer cells, HSC-4. RNA Seq analysis reveals that many of the downstream targets of IL6 pathway including JUN and FOS, which are the components of AP1 are up-regulated. At the cellular level, it is observed that co-localization of SSTP1 and IL6Rα on the cell surface and co-localization of SSTP1, IL6Rα and Rab5C in the cytoplasm suggesting the endosome mediated internalization that follows receptor activation. The biochemical evidences showed that STAT3 phosphorylation (pSTAT3 Y705) by JAK activity is considerably reduced, while JNK activity and cJUN phosphorylation is up-regulated with increased transcriptional activity of AP1. We did not observe any specific change in MAPK pathway and PI3K pathway, which are also known to be activated downstream of IL6. The use of specific inhibitors for IL6 (LMT-28), JAK (Ruxolitinib), gp130 (SC-144) and JNK (SP600125) as well as Anti-IL6Rα showed that SSTP1-mediated cJUN phosphorylation and apoptosis induction are dependent on IL6, IL6Rα, gp130 and JNK activities.

The present invention will be described herein after with reference to a number of non-limiting Examples.

### Experiment 1

Identification of Antitumor Peptides from Skin Secretion of Indosylvirana Aurantiaca

The present invention uses Frog (Indosylvirana aurantiaca) skin secretion to construct cDNA library of secreted peptides by shotgun cloning. 14 mature peptides were identified and were chemically synthesized to check the antitumor activity with the help of MTT assay. The peptide Temporin1Dau1 was identified and selected for further studies due its low IC50 value. This peptide is referred as SSTP1. The physico-chemical properties of SSTP1 including molecular weight, net charge, hydrophobicity etc, are the features required for antitumor activities. The helical wheel projection of SSTP1 showed a spatial separation of hydrophilic residues and hydrophobic residues, which is a determinant for amphipathicity, is the quality required for antitumor activity. The antitumor activity of SSTP1 was checked in comparison to another peptide where the hydrophobic residues were replaced with Arg, whose sequence is FLPRRISARTSLFPKRGK-NH2 (SEQ ID NO: 2) the peptide is SSTP2, which has less helicity hydrophobicity, amphipathicity, and high positive charge, Thus, the in silico analysis suggested that SSTP1 could be a potent antitumor peptide, while SSTP2 might serve as a negative control.

### Experiment 2.1

SSTP1 Induces Apoptosis in Oral Cancer Cells

Evaluation of the cytotoxicity of SSTP1 and SSTP2 was performed using MTT assay in an oral cancer cell line, HSC-4 at various concentrations of the respective peptides. FIG. 1a represents the % inhibition of viability as assessed by MTT assay, which indicates that SSTP1 induces growth inhibition with an IC50 value of 10.22 μM, while SSTP2 is ineffective in inducing cytotoxicity.

### Experiment 2.2 Annexin/PI Staining

To check whether the cytotoxic effect is due to apoptosis, Annexin/PI staining was performed, with the population with Annexin alone (early apoptotic), Annexin/PI dual stained (late apoptotic) and PI alone (dead cells) were quantified by FACS. The results revealed a 3-fold increase in the early apoptotic population upon SSTP1 treatment as shown in FIGS. 1b and c.

### Experiment 2.3 Caspase Cleavage

Further analysis showed the active cleavage of Caspase 3, 7 and 9, while Caspase 8 was unaffected by SSTP1 treatment, however PARP cleavage within 16 hours of treatment with SSTP1 was observed. The cells treated with SSTP2 neither showed an increase in apoptotic population nor activation of any Caspases as show in FIGS. 1d and e. The above results suggest that the cytotoxicity induced by SSTP1 is through the induction of apoptosis by mitochondrial pathway.

### Experiment 3

The Induction of Apoptosis by SSTP1 Involves a Non-Membranolytic Activity

a. Our live imaging showed that SSTP1 is binding to the membrane, and it starts internalizing within 30 min. Though at a lower rate, SSTP2 was also binding to the membrane and internalizing. When we repeated the experiments on ice, where cells are not physiologically active, SSTP1 was not appreciably internalized as observed at 37° C., while internalization of SSTP2 was not affected (FIG. 2a). Since these results suggested the involvement of a receptor-mediated internalization for SSTP1, we decided to check whether it is clathrin-dependent mechanism, using labelled transferring (FIGS. 2b, c and d). Since SSTP1 was not internalized along with transferrin, as suggested by the negligible Mander's overlap, the entry of SSTP1 is probably through receptor-dependent clathrin-independent endocytosis. We further investigated the genes and pathways activated by SSTP1 treatment, which might give insight to its membrane receptors.
b. FIG. 2 illustrates SSTP1 is internalized by active uptake where in specific 2(a) illustrates the live HSC-4 cells were incubated with Mito Tracker Deep Red and incubated for 10 min. Excess dye was washed off, and then the respective peptides were added, which were labelled with Alexa Fluor-488, and incubated either on ice or at 37° C. Images were acquired at the indicated time intervals. 2(b) shows the cells were treated with Alexa Fluor-488 labelled SSTP1 for indicated time and Alexa Fluor-568-labelled transferrin was added and incubated for one min, the cells were washed, fixed and imaged. 2(c) illustrates the cells were treated with SSTP1-Alexa Fluor-488 for 20 min; transferrin-568 was added to it and incubated for one minute. Cells were washed to remove unbound SSTP1 and transferring. Live imaging was performed after 2 minutes. 2(d) the extent of co-occurrence for panel b was measured and plotted as Mander's overlap. The median value is shown in the graph of FIG. 2.

### Experiment 4

RNA-Seq Analysis Suggests the Involvement of IL6/IL6Ru Pathway in SSTP1-Induced Apoptosis.

In order to identify the target genes of SSTP1, an RNA-Seq on Illumina platform was performed using cells treated with SSTP1 in comparison to SSTP2. Out of the 34 to 37 million reads obtained, 31 to 34 million reads were mapped onto the indexed Human reference genome GRCh38.p7. the differential gene expression analysis identified 208 up-regulated genes and 118 down-regulated genes as shown in FIG. 3a The RNA-Seq results were validated by 12 up-regulated genes and 4 down-regulated genes by RT-PCR and q-PCR as shown in FIG. 3b. Several of the differentially expressed genes were related to apoptosis.

### Experiment 5

SSTP1 Binds to IL6Rα on the Cell Membrane

To check whether SSTP1 activates the IL6/IL6R pathway by binding to IL6Rα, we checked the co-localization of SSTP1 and IL6Rα on cell membrane. We observed several foci on the cell surface with co-localization of SSTP1 and IL6Rα, while co-localization of SSTP2 to IL6Rα was rare as shown by the Pearson's coefficient (FIGS. 4a and b). In order to rule out a nonspecific interaction of SSTP1 to a random surface receptor, we performed a co-immunofluorescence analysis of SSTP1 with several surface molecules like tubulin α5, β-tubulin, DSG3, DLL1 and GRPR, which did not show co-localization with the peptide. To visualize the IL6R-dependent SSTP1 endosomal up-take, we used an early endosome marker, Rab5C. We observed the co-existence of SSTP1 and IL6Rα within early endosomes, marked by Rab5C (FIGS. 4c and d). The physical interaction of SSTP1 with IL6Rα was stronger than that of SSTP2, as revealed by the pull-down assay using biotin-labelled peptides FIG. 4e). The physical interaction of SSTP1 and IL6Rα was also ascertained using FRET analysis (FIG. 4f).

FIG. 4 illustrates SSTP1 interacts with IL6Rα and mediates the uptake of SSTP1 specifically FIG. 4(a) discusses HSC-4 cells were treated with Biotin labelled SSTP1 or SSTP2 (10 μM) for 30 minutes and then fixed and probed without permeabilization using mouse IL6Rα. 4(b) illustrates the co-localization of SSTP1 or SSTP2 to IL6Rα was analyzed for 11 cells and the Pearson's correlation was plotted. 4(c) illustrates cells were treated as in panel (a) and fixed and permeabilized to probe for IL6Rα, SSTP1 and Rab5c. The arrow mark indicates the co-localization. The scales bars represent 4 μm. 4(d) illustrates the Mander's overlap in SSTP1 foci were quantified for 10 cells. The average values for each cell is plotted. 4(e) illustrates the pull down assay using SSTP1-biotin and probed for IL6Rα. 4(f) illustrates the FRET analysis to show the SSTP1 binding to IL6Rα.

### Experiment 6

In Silico Analyses Suggest that SSTP1 and SSTP2 Exert Differential Effects on Active Sites of IL6/IL6Rα/Gp130 Complex

a. To get insight into dynamics of peptide binding, we performed some in silico analyses. As the peptides' crystal structures were not known, we used the 3D structure of another peptide from the same family, Temporin-1Ta, as a template to predict the structures of SSTP1 and SSTP2. Since signalling depends on the formation of the active complex of IL6 and its receptors, we docked the peptides to the reported crystal structure of the active hexameric complex. The analysis predicted stronger interaction for SSTP1 than SSTP2 (with scores −985.038 and −789.990 for SSTP1 and SSTP2, respectively). Dynamic simulations for the structures were performed, and the resulting stable conformations with the least energy were analyzed further. Both SSTP1 and SSTP2 bind to the interface between IL6 and IL6Rα, but in a different orientation (FIG. 5a).

The superimposition of the peptide-bound complexes to the unbound complex showed that a unique rearrangement occurs in the IL6/IL6R interface of SSTP1-bound complex, specifically in a region that forms an interface between gp130 of the other trimer (FIG. 5b). The hydrogen bonds formed between Arg30 and Glu297; Arg182 and Ser247; as well as Lys54 and Gln209 were disrupted when SSTP1 or SSTP2 binds to the complex (FIG. 5c). On further analysis, it was found that the significant unique change observed in the SSTP1-bound complex is in the interface region constituted by Phe153 and Asn155 of IL6Rα and Lys54, Glu55, Ala56, Leu57 and Asn61 of IL6 (FIG. 5c). There was a difference in the values for accessible surface area, buried surface area and solvation energy for these residues in SSTP1-bound 1P9M in comparison to 1P9M and SSTP2-bound 1P9M, so that the conformation of these residues was significantly altered.

### Experiment 7

SSTP1 Down-Regulates JAK/STAT Pathway and Activates JNK/AP1 Pathway

a. There are evidences to show that IL6/IL6R pathway leads to the activation of JAK/STAT, JNK/AP1, MAPK and PI3K/NF-κB pathways (12-15). The phosphorylated forms of STAT1 (Y701) and STAT3 (Y705) were probed for western blot to see the JAK/STAT pathway activation. Another phosphorylation of STAT3 at S727, which is reported to be independent of JAK activation, was also probed. Even though SSTP1 did not affect STAT1 (Y701) phosphorylation significantly, it drastically reduced STAT3 (Y705) phosphorylation. On the other hand, SSTP2-treatment induced a mild up-regulation of STAT1 phosphorylation with simultaneous mild down-regulation of STAT3 phosphorylation (FIGS. 6a and b). At the same time, there was a slight up-regulation of pSTAT3(S727) by both SSTP1 and SSTP2. Phosphorylation of JNK1 was markedly up-regulated by SSTP1 than SSTP2. Correspondingly, there was increased level of pc-JUN when treated with SSTP1 than SSTP2. At the same time, pJNK2 levels were up-regulated by both SSTP1 and SSTP2. While both the peptides down-regulated pMAPK levels, pAKT levels were unaffected. All these biochemical analyses point out that SSTP1 preferentially down-regulates JAK-mediated-phosphorylation of STATs and MAPK, simultaneously up-regulating phosphorylation of JNK1 and cJUN.
b. To investigate whether the JNK-mediated activation is regulating AP1 activity, a luciferase AP1 reporter assay was performed, which showed that AP1 is activated by SSTP1 in comparison to SSTP2 as shown in figure FIG. 6c.

### Experiment 8

SSTP1-Mediated Induction of Apoptosis Depends on the Activation of IL6/IL6Rα, Gp130 and JNK

a. Inhibitors for IL6 (LMT-28), JAK (Ruxolitinib), gp130 (SC-144) and JNK (SP600125) as well as Anti-IL6Rα were used to check the dispensability of each of these molecules in inducing apoptosis by SSTP1. The addition of these inhibitors blocked the phosphorylation of STAT1 and STAT3 (FIGS. 7a and b). The SSTP1-mediated up-regulation of pJNK was not appreciably affected by either of these inhibitors, while pcJUN up-regulation was inhibited by only SP600125 (FIGS. 7a and b). At the same time, SC-144, the inhibitor for gp130, and an antibody for IL6Rα significantly reduced phosphorylation of STATs, JNK and cJUN (FIGS. 7c and d). The SSTP1-induced growth inhibition of cancer cells was significantly inhibited by anti-IL6Rα and SC144, whereas LMT28 was ineffective (FIG. 7e). LMT28 binds to the interface between IL6 and gp130 to block the activity of IL6. Since SSTP1 binds to a different site on the IL6/IL6Rα/gp130 complex, i.e., the interface of IL6 and IL6Rα, there is no competitive inhibition when we add LMT28.
b. FIG. 7 illustrates the SSTP1 induced pc JUN activation depends on JNK and on gp130 and IL6Rα, where 7(a) illustrates HSC-4 cells treated with the peptides and inhibitors were used for western blot analysis of the indicated molecules. 7(b) illustrates densitometric analysis was done for the bands of (a) and relative phosphorylation is plotted. 7(c) illustrates the cells treated with the peptides and IL6Rα antibody and gp130 inhibitors were used for western blot analysis of the indicated molecules. 7(d) illustrates the denstiometric analysis was done for the bands (c) and relative phosphorylation is plotted. 7(e) illustrates the growth inhibition analyzed by MTT assay after 4 h treatment with SSTP1 (10 μM) with or without LMT-28 (60 μM), Anti-IL6Rα (10 μg/ml) or SC144 (40 μM).

### Experiment 9

Growth Inhibition by SSTP1 Depends on IL6Rα Levels

The evaluation of the mechanism of induction of growth inhibition by SSTP1 showed that it depends on the modulation of IL6/IL6R pathway by binding to IL6Rα. This suggested that the peptide has better growth inhibitory property on IL6Rα-overexpressing cell lines. Further, the growth inhibitory property of SSTP1 on different cell lines with different levels of IL6Rα was investigated. Six cancer cell lines of different origin were evaluated for their IL6Rα expression in comparison to HSC-4 by western blot (FIGS. 8a and b). The results showed that there is a significant up-regulation of IL6Rα expression in all the six cell lines compared to HSC-4 which were treated with 10 μM SSTP1, and MTT assay was performed to find the growth inhibition. As expected, there was a drastic increase in the growth inhibitory potential in all the six cell lines (FIG. 8c). Importantly, there was 91% growth inhibition in MDA-MB-231, which is a triple negative breast cancer cell line. The MDA-MB-231 was selected and determined the IC50 value for SSTP1, which was reduced to 4.5 μM (FIG. 8d). Further, the SSTP1-induced cell death in MDA-MB-231 cells depended on the activation of JNK and cJUN rather than the reduction of STAT3 activity (FIGS. 8e and f). Since many of the bioactive peptides induce hemolysis, we evaluated the hemolysis induced by SSTP1 (FIG. 8d). The growth inhibitory response of SSTP1 on MDA-MB-231 cells and induction of hemolysis by SSTP1 on human RBCs are summarized in FIG. 8d. Notably, the hemolytic activity at 5 μM concentration of SSTP1 was only 0.25% which shows that administration of SSTP1 at IC50 concentrations will not induce any hemolysis in the body, the trypan blue exclusion of leukocytes after 48 h of treatment with the peptide showed that there is no adverse effect of the peptide on the viability of these cells (FIG. 8g).

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

1. An anticancer peptide comprising the amino acid sequence as,

| | a. |
|---|---|
| (SEQ ID NO: 1) | |
| | FLPLLISALTSLFPKLGK. |

2. A peptide as claimed in claim 1 is a derivative of peptide SSTP1.

3. A peptide as claimed in claim 1 wherein the peptide has a length of 18 amino acids.

4. A peptide as claimed in claim 1 wherein the peptide selectively induces apoptosis in IL6Rα overexpressing cells.

5. The IL6Rα overexpressing cells as claimed in claim 4 are triple negative breast cancer cells.

6. A peptide as claimed in claim 1 is a cDNA construct.

7. A pharmaceutical composition comprising therapeutically effective amount of the peptide as claimed in claim 1 as its active ingredient along with the pharmaceutically acceptable peptide stabilizer, carrier and excipients.

8. A pharmaceutical composition as claimed in claim 7, where the active ingredient is a derivative of peptide SSTP1.


SEQUENCE LISTING (2 sequences)

SEQ ID NO: 1 - protein, 18 aa, Indosylvirana aurantiaca
FLPLLISALTSLFPKLGK

SEQ ID NO: 2 - protein, 18 aa, Artificial Sequence
  Description of Artificial Sequence Synthetic peptide
  C-term NH2
FLPRRISARTSLFPKRGK